(19) United States
(12) Patent Application Publication
(10) Pub. No.: US 2011/0213464 A1
(43) Pub. Date: Sep. 1, 2011

(54) INJECTION OF FIBRIN SEALANT IN THE ABSENCE OF CORTICOSTEROIDS IN SPINAL APPLICATIONS

(76) Inventors: Steven I. Whitlock, Austin, TX (US); Brian D. Burkinshaw, Pflugerville, TX (US); Kevin Pauza, Tyler, TX (US); John L. Wheeler, Austin, TX (US); Kevin Thorne, Austin, TX (US)

(21) Appl. No.: 12/910,237

(22) Filed: Oct. 22, 2010

Related U.S. Application Data
(63) Continuation-in-part of application No. 11/205,760, filed on Aug. 17, 2005.
(60) Provisional application No. 60/623,600, filed on Oct. 29, 2004.

(51) Int. Cl. A61F 2/44

(52) U.S. Cl. 623/17.16

(57) ABSTRACT

A method and kit for treating a disc that is leaking nucleus pulposus through at least one defect in the annulus fibrosus. The method includes injecting a fibrin sealant into the disc to reduce at least a portion of the at least one defect, wherein the fibrin sealant injected into the disc comprises fibrinogen and an activating compound, wherein at least a portion of the fibrin forms after injection, with the proviso that a corticosteroid is absent from the fibrin sealant injected into the disc.

### CROSS-REFERENCE TO RELATED APPLICATIONS

This application is a continuation-in-part of U.S. application Ser. No. 11/205,760, filed Aug. 17, 2005, which claims priority to U.S. provisional application No. 60/623,600, filed on Oct. 29, 2004. All of the above applications are hereby incorporated by reference.

### BACKGROUND OF THE INVENTION

The present invention relates generally to the use of fibrin sealant whereby the sealant is delivered such as by injection to the spinal area.

Fibrin sealants, and glues, are well known and are used extensively in various clinical settings. Such sealants are indicated as adjuncts to hemostasis in surgeries when control of bleeding by conventional surgical techniques, including suture, ligature, and cautery is ineffective or impractical. In these cases, the sealant was applied topically.

Recently, fibrin sealant that included a corticosteroid was used to treat disc problems such as fissures in the annulus fibrosus. In this regard, U.S. Pat. No. 6,468,527 discloses that the composition was injected into a disc (an intra-discal injection) to treat disc problems.

The pathogenesis of chronic, low back pain (CLBP) may be due to intervertebral disc (IVD) degeneration. The degeneration of the IVD may be a complex, age-related process influenced by several poorly understood genetic, biologic and mechanical factors. These factors may lead to reduced cell nutrition, cell death and increased concentrations of proinflammatory cytokines and proteolytic enzymes. This combination may adversely influence the reparative function of normal IVD cells and favor catabolic tissue degeneration. Progressive catabolic tissue degradation may result in reduced proteoglycan and water content within the nucleus pulposus, increased fibrosis of the nucleus pulposus, decreased disc height and increased load sharing by adjacent vertebral elements. Disc degeneration may correlate to CLBP in the presence of specific anatomic features including, for example, concentric and radial fissures of the anulus fibrosus and posterior disc protrusion. More extensive disc degeneration may also compromise adjacent spinal structures and elicit both back and leg pain through vertebral facet arthropathy, foraminal stenosis and spondylolisthesis.

Anatomical changes leading to IVD degeneration develop early in life (<20 years of age). The process may be initiated by a dramatic decrease in the concentration of physiologic vessels in the cartilaginous endplates that support vascular nutrition of the IVD. As a consequence of these vascular reductions, the populations of notochordal cells may gradually disappear to be replaced by reduced concentrations of fibrochondrocytes. The reductions in vascular nutrition and cell population may adversely influence the cellular synthesis of proteoglycans. The gradual reductions in proteoglycan content may inhibit the ability of the disc to maintain the hydration required for normal load distribution and result in inappropriate stress concentrations along both the endplates and the anulus fibrosus. In approximately 20% of this age demographic, localized stress concentration sites produce cracks in the cartilaginous endplates and concentric and radial tears in the anulus fibrosus of the IVD.

As patients age, continued reductions in vasculature and the accumulation of chronically elevated concentrations of proinflammatory cytokines and catabolic enzymes accelerate catabolic tissue degeneration. This promotes the formation, growth and accumulation of structural disruptions of the IVD. The prevalence of internal disc disruptions (IDD) ranges between 50% and 90% for adults between the ages of 30 and 60. Pathogenic sensation of chronic low back pain may manifest when the internal disc disruptions of the anulus fibrosus (IDD) expose nociceptive nerves contained within its peripheral layers. Pain originates from the interaction of these nociceptive nerves with the hyperalgesic inflammatory cytokines concentrated within the nucleus pulposus. Pathogenic IDD is considered as a distinct source of axial back pain as it presents without radiographic evidence of severe degenerative disc disease, such as significant disc height loss, facet arthropathy, foraminal stenosis, osteophyte formation and segmental instability. Commonly referred to as discogenic pain, pathogenic IDD represents a common source of CLBP (˜39%) in the working age population.

### SUMMARY OF THE INVENTION

In the practice of the present invention, fibrin sealant is injected into the spinal area of a human being. The sealant comprises fibrinogen and an activating compound such as thrombin, which form fibrin when mixed. However, corticosteroids are excluded from the fibrin sealant. It has been found that this composition provides surprisingly superior results relative to fibrin sealant compositions containing a corticosteroid. Calcium ions, such as supplied from calcium chloride, may be included in the fibrin sealant. The fibrin may optionally include one or more additives, such as various biological and non-biological agents.

In one broad respect, this invention is a method of treating a disc that is leaking nucleus pulposus through at least one defect in the annulus fibrosus, comprising: injecting a fibrin sealant into the disc to reduce at least a portion of the at least one defect, wherein the fibrin sealant injected into the disc comprises fibrinogen and thrombin, wherein at least a portion of the fibrin forms after injection, with the proviso that a corticosteroid is absent from the fibrin sealant injected into the disc. The defect can be a tear of the annulus fibrosus, a fissure in the annulus fibrosus, and the like. This treatment serves to reduce the amount of material from the nucleus fibrosus that leaks through the defect(s) in the annulus fibrosus. Advantageously, injection of the fibrin sealant can also serve to restore normal disc height and physiologic hydrostatic pressure, key components to disc health. It should be understood that normal physiologic hydrostatic pressure can vary from person to person, and that the treatment may produce near-normal hydrostatic pressure. As used herein, normal physiologic pressure encompasses this range of pressures. In one embodiment, neither the nucleus pulposus nor the annulus fibrosus has been heated in the body to stiffen the disc either prior to or concurrent with the injection, such as discussed in for example U.S. Pat. No. 6,095,149. In one embodiment, in the practice of this invention the nucleus pulposus has not been removed by surgery, such as in the case of a total or partial discectomy or by nucleoplasty for a herniated disc.

In another broad respect, this invention is a method of treating a human back, comprising injecting a fibrin sealant into a disc to seal at least one defect of an annulus fibrosus, wherein the fibrin sealant comprises fibrinogen and an activating compound such as thrombin, wherein the fibrinogen and thrombin forms at least a portion of the fibrin after injection, and wherein the fibrin sealant does not include a corticosteroid.

In another broad respect, this invention is a method of treating a human back, comprising providing a mixture of fibrinogen and thrombin within a human disc to treat at least one defect of an annulus fibrosus, and wherein a corticosteroid is absent from the mixture. The mixture may be provided into the disc by injection or otherwise.

This invention also includes a kit including components used to inject the fibrin sealant. The kit may comprise fibrinogen, such as freeze-dried fibrinogen, thrombin such as freeze-dried thrombin, and a needle for injecting the sealant such as a spinal needle including for example a curved spinal needle. A spinal canula may alternatively be used. The kit excludes corticosteroid. The kit may exclude a device to provide thermal energy to a disc. The kit can optionally include contrast agent and other additives. A single, dual or multi-barrel syringe, or other fibrin sealant delivery device, may be included in the kit. The fibrin sealant can be delivered using a convention single lumen needle, or through a bilumen or multilumen needle. If a bilumen needle is used, each component can be delivered through a separate lumen. In one embodiment, a bilumen needle or multilumen needle can be used that allows contact of the fibrinogen component and the thrombin component at the tip of the needle. Alternatively, sequential addition of the fibrinogen component followed by injection of the thrombin or other enzyme component can be used, and these injections can occur in the same needle, multiple needles, or in a bilumen or multilumen needle.

In another broad respect, this invention is a process for forming a kit, comprising: providing a fibrinogen component, an activating compound, an introducer needle, and a spinal needle or synthetic catheter, wherein the kit excludes corticosteroid and wherein the kit excludes a device to provide thermal energy to a disc.

Advantageously, the method and kit of this invention facilitate extended pain relief for patients with leaky disc syndrome, wherein for example nucleus pulposus leaks out of the disc through defects (e.g. tears or fissures) in the annulus fibrosus. Surprisingly, it has been found that the use of fibrin alone provides unexpectedly superior results to injections of fibrin sealant that includes a corticosteroid. The present invention provides unexpectedly superior results to the method set forth in U.S. Pat. No. 6,468,527, which discloses the injection of fibrin sealant containing a corticosteroid.

### BRIEF DESCRIPTION OF THE DRAWINGS

FIG. 1 is a cross-sectional view of a vertebral body at the disk space exhibiting annular fissures that may be treated according to one embodiment of the present invention.

FIG. 2 is a schematic representation of the trans-foraminal space into which the improved sealant may be injected according to one embodiment of the present invention.

FIGS. 3 and 4 show graphs of the VAS scores from example 3.

### DETAILED DESCRIPTION OF THE INVENTION

The fibrin sealant of the present invention comprises a fibrinogen component and an activating compound such as thrombin that converts fibrinogen to fibrin. The sealant may contain one or more other components. The fibrin sealant is injected into, for example, the disc to seal fissures and tears in the annulus fibrosus. Defects in the annulus fibrosus are commonly diagnosed, currently, using MRI scans and discograms. This can treat both discogenic low back pain and radiculopathy leg pain when injected into the lumbar intervertebral disc.

The fibrinogen used in the practice of this invention includes any fibrinogen that will form fibrin in a human body. Fibrinogen is frequently available in freeze-dried form, and must be reconstituted prior to use. The fibrinogen can also be frozen or fresh. The fibrinogen can be autologous (from the patient to be treated), human including pooled human fibrinogen, recombinant, and bovine or other non-human source such as fish (e.g., salmon and sea trout). The fibrinogen is used in an amount suitable for the given treatment, patient, and so on. The freeze-dried fibrinogen can be reconstituted using, for example, saline, a saline solution containing aprotinin, a saline solution containing calcium ions (Ca⁺²) such as may be supplied from calcium chloride, a saline solution containing one or more other additives such as a local anesthetic, or a solution containing combinations of additives.

Thrombin is typically the enzyme used which serves to change fibrinogen to fibrin. However, other enzymes can be used to convert fibrinogen to fibrin, such as those derived from snake venom (e.g., batroxobin), or spider venom as is known in the art. As used herein, “activating compound” refers to a compound that causes fibrinogen to form fibrin, and this term includes thrombin, batroxobin, and so on. Thrombin is available commercially, typically in its freeze-dried form. Freeze-dried thrombin must be reconstituted prior to use. The thrombin can also be frozen or fresh. Thrombin can be autologous, from a human or pooled human supply, bovine, fish (such as salmon) or other non-human fibrinogen-cleaving enzyme source such as various arachnids and other venomous species. The thrombin or enzyme is used in any amount which facilitates changing the fibrinogen to fibrin, as is known to one of skill in the art. The thrombin can be reconstituted using saline, a saline solution containing calcium ions, a saline solution containing one or more other additives such as a local anesthetic, or a solution containing calcium ions and one or more additives.

Additional additives may be employed in the fibrin sealant such as, but not limited to: antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; vitamins and other nutritional supplements; hormones; glycoproteins; fibronectin; peptides including polypeptides and proteins; interferons; cartilage inducing factors; protease inhibitors; vasoconstrictors, vasodilators, demineralized bone or bone morphogenetic proteins; hormones; lipids; carbohydrates; proteoglycans such as aggrecan (chondrotin sulfate and deratin sulfate), versican, decorin, and biglycan; antiangiogenins; antigens; DBM; hyaluronic acid and salts and derivatives thereof; polysaccharides; cellulose compounds such as methyl cellulose, carboxymethyl cellulose, and hydroxy-propylmethyl cellulose and derivatives thereof; antibodies; gene therapy reagents; genetically altered cells, stem cells including mesenchymal stem cells with transforming growth factor, and/or other cells; cell growth factors to promote rehabilitation of damaged tissue and/or growth of new, healthy tissue such as BMP7 and BMP2; type I and II collagen; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; and nutrients such as, for example, glucose or other sugars. However, it is foreseeable that any of these additives may be added to the fibrin sealant separately or in combination. One or more of these additives can be injected with the fibrinogen and activating compound, or alternatively one or more of these components can be injected separately, either before or after the fibrin sealant has been injected.

For solutions containing an incompletely water-soluble additive(s), an anti-caking agent such as, for example, polysorbate, may be added to facilitate suspension of this component. Glycol may be inappropriate for use as an anti-caking agent in the instant invention.

It should be appreciated that fibrin formation begins immediately on contact of the fibrinogen and thrombin, such as in the Y-connector of a dual syringe. The term “injecting” of fibrin sealant as used herein thus encompasses any injection of components that form fibrin in the disc, including circumstances where a portion of the components react to form fibrin due to mixing prior to contact with or actual introduction into the disc. It is also within the scope of this invention to sequentially inject the components of the fibrin sealant into the disc, such as by injecting the thrombin component followed by the fibrinogen component, or by injecting the fibrinogen component followed by the thrombin component. Likewise, the fibrinogen component and the thrombin components can be each intermittently injected into the disc.

It should also be appreciated that the point, or points, of injection (e.g., at the tip of a spinal needle) can be within the annulus fibrosus or in the nucleus pulposus. If the injection occurs in the nucleus pulposus, the injected components may form a patch at the interface between the nucleus pulposus and the annulus fibrosus, or, more commonly, the components flow into the defect(s) (e.g., fissures) of the annulus fibrosus and potentially “overflowing” into the interdiscal space. In practice, over-pressurizing the disc by injecting the components into the disc should be avoided.

The fibrinogen and activating compound are injected in amounts effective to seal a given defect of the disc, as is apparent to one of skill in the art. The amount of activating compound such as thrombin can be varied to reduce or lengthen the time to complete fibrin formation. In general, the higher level of thrombin per unit amount of fibrinogen, the faster fibrin formation occurs. If slower fibrin formation is desired, then less thrombin is used per unit fibrinogen. The use of calcium ions (such as from calcium chloride) in one or both of the component solutions will affect the strength of the fibrin so formed, with increasing amount of calcium ions increasing the strength of the fibrin clot. Generally, for a composition comprising fibrinogen that is an aqueous solution, it is believed that from about 3 mL to about 5 mL of such composition is sufficient to be an effective fibrin sealant. However, depending on the use of the composition, the dosage can range from about 0.05 mL to about 40 mL.

Fibrin sealants mimic the final stage of the natural clotting mechanism. Typically, such sealants entail the mixing of a fibrinogen component with an activating enzyme such as thrombin. Thrombin is an enzyme that exists in blood plasma which causes the clotting of blood by converting fibrinogen into fibrin. In normal practice, the components of the fibrin sealant are reconstituted separately, from a freeze-dried state, prior to use. However, the use of samples prepared from a frozen state or a fresh state is also acceptable. To increase biocompatibility of the sealant with host tissue, various components may be supplied endogenously from host body fluids. Combining the reconstituted components produces a viscous solution that quickly sets into an elastic coagulum. A method of preparing a conventional fibrin sealant is described by J. Rousou, et al. in Journal of Thoracic and Cardiovascular Surgery, vol. 97, no. 2, pp 194-203, February 1989. Cryoprecipitate derived from source plasma is washed, dissolved in buffer solution, filtered and freeze-dried. The freeze-dried fibrinogen is reconstituted in a fibrinolysis inhibitor solution containing, for example 3000 KIU/ml of aprotinin (a polyvalent protease inhibitor which prevents premature degradation of the formed fibrin). The solution is stirred and heated to a temperature of about 37° C. Each solution (the thrombin and fibrinogen solutions) is drawn up in a dual barrel syringe and mounted on a Y-connector to which a needle is attached for delivery of the combined solution. (See, e.g. the Duploject® device, from ImmunoAG, Vienna, Austria). Thus, mixing of the components only occurs during the delivery process which facilitates clot formation at the desired site of application only. The components should be injected sufficiently quickly to avoid the passage becoming blocked due to fibrin formation in the needle and/or Y-connector.

In one embodiment, the mixing of the fibrin sealant components at least partially occurs in the Y-connector and in the needle mounted on a Y-connector, with the balance of the clotting occurring in the disc. This method of preparation facilitates the formation of a fibrin clot at the desired site in the disc during delivery, or immediately thereafter. Calcium ions may be included in the fibrin sealant to be injected to modify the composition of the so-formed fibrin and resulting strength of the clot.

In one embodiment, about 75-105 mg/mL of freeze-dried fibrinogen is reconstituted according to conventional methods, and about 45-55 mg/mL thrombin component is reconstituted separately from a freeze-dried state according to the methods and compositions of the present invention. Freeze-dried fibrinogen and freeze-dried thrombin are available in kit-form from such manufacturers as Baxter under names such as TISEEL®. These two fibrin sealant components can be prepared for example in about 2 mL samples each to yield approximately 4 mL of total sealant (reconstituted fibrinogen plus reconstituted thrombin).

While several methods and compositions may be used for preparing the freeze-dried thrombin for use in the invented fibrin sealant, one method is providing about 45-55 mg/mL of freeze-dried thrombin and mixing it with a reconstituting solution. The reconstituting solution may optionally further comprise about 0.1-100 milligrams of another additive described herein (e.g., local anesthetic) and/or calcium ions. The calcium ion solution (e.g.: calcium chloride) concentration can be, for example, 1-100 millimoles/mL, and in one embodiment 4-40 millimoles/mL. If employed, the calcium +ion concentration should be sufficient to further the polymerization reaction that forms a durable fibrin sealant clot. A preservative-free reconstituting solution may be desirable, but is not required.

A contrast agent may be used in conjunction with the injection of the fibrin sealant. The contrast agent may be injected prior to injection of the fibrin sealant. Alternatively, the contrast agent is included in the fibrinogen component or thrombin component that is injected into the disc. Contrast agents and their use are well known to one of skill in the art.

Alternative amounts and concentrations of fibrinogen and thrombin may be used to form the desired fibrin sealant clot in the body. For example, as discussed above, varying the fibrinogen and/or thrombin amount/concentration may be done to vary the viscosity and the “setting time” of the combined fibrinogen and thrombin components. Likewise, varying fibrinogen may change the density of the combined components, which may be important for controlling flow through a long conduit such as a catheter into the body. Varying thrombin may vary the polymerization time of the components, which may be important for controlling the time at which the clot forms for ensuring the components set-up at the proper site and time in the body rather than setting-up prematurely.

When acquired in freeze-dried form, the thrombin and fibrinogen need to be reconstituted for use. The thrombin reconstituting solution (e.g., a saline based solution), optionally containing one or more additives, can be prepared in a single vial prior to mixing with the freeze-dried thrombin. This component of the fibrin sealant may then be provided to users in a reconstituted state, or in two uncombined vials containing freeze-dried thrombin and a premixed reconstitution solution. Mixing of the contents of the two vials may be performed at any point up to, and including, the time at which the fibrin sealant (or its components) is injected into the patient. Reconstitution of the fibrinogen solution can be accomplished according to conventional methods. For example, the fibrinogen component may be reconstituted in an aprotinin saline solution which optionally contains additives such as, for example, a local anesthetic. If desired, the thrombin or the fibrinogen or both can be reconstituted using a saline solution that contains one or more additives. All solutions are brought to a temperature of about 37° C. Preferably, the thrombin is combined with the fibrinogen solution using the dual-syringe injection procedure described herein to form a single sealant composition which is injected into a patient. The instant invention provides a vehicle for the delivery of the sealant that conveys the sealant to the precise area of the back, seals any annular fissures, and holds the fibrin in place via the elastic coagulum. In addition, the biodegradable nature of the formed fibrin clot minimizes or eliminates the need for invasive surgical removal following the effective period of use. Therefore, an advantage of the sealant and method of application is the ability to provide a minimally invasive means of accomplishing localized, prolonged sealing of defects (e.g., fissures) in the annulus fibrosus, and if an additive is in the sealant, time-released additive delivery.

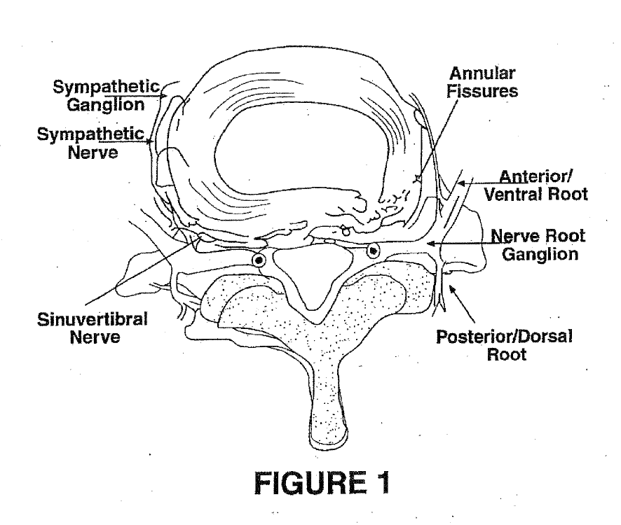

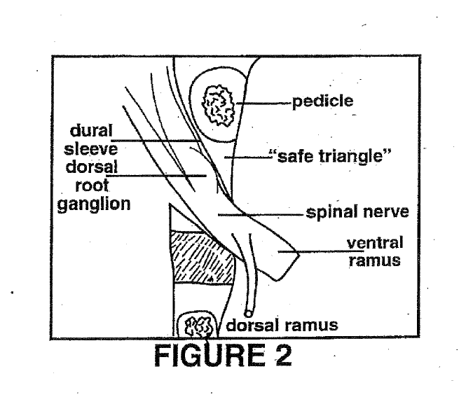

The fibrin sealant may be injected into the disc or other body area using procedures well known to one skilled in the art. Typically, an introducer needle is inserted into the intra-discal space with the tip being positioned close to the defect in the annulus fibrosus. A finer gauge needle (made of, e.g., stainless steel and capable of puncturing the annulus fibrosus) is then inserted into the introducer needle. The fibrin sealant is injected through the finer gauge needle. Alternatively, a catheter made from a synthetic polymer can be used. With either a finer gauge needle or a catheter made of synthetic polymer, the needle or catheter can be advanced through the introducer needle and into the nucleus pulposus. Alternatively, the needle or catheter can be advanced up to the tip of the introducer needle, but not far as to go beyond the tip of the introducer needle. This can have the advantage of precisely positioning the point of injection, particularly since a polymeric catheter could bend in the nucleus propulsus thereby become mis-positioned. Likewise by positioning the introducer needle at the desired point of injection as an initial matter, the fibrin sealant can be injected quickly to expedite the procedure, which is a benefit to the patient. In general, the fibrin sealant of this invention is injected into the disc, the epidural space, the zygaphysical (2-joint) joint, the vertebral canal, and/or thecal sac. With respect to an injection of fibrin sealant into a disc, an intra-discal injection serves to create a fibrin matrix which seals the disc from leaking material from the nucleus into the area outside the disc. For example, the fibrin sealant can be delivered by fluoroscopic transforaminal lumber epidural or intra-discal injection, such as described in U.S. Pat. No. 6,468,527. For the treatment of back injuries such as these, the fibrin sealant is injected into the nucleus pulposus, shown in FIG. 1, to fill any fissures or voids of the annulus fibrosus, to seal the bone end plates to the disc, increase pressure of the disc, and to increase the height of the disc space. In general, the fibrin sealant is injected at a location near the defect in the annulus fibrosus. Typically the fibrin sealant will flow into the fissures in the annulus fibrosus, and some fibrin sealant may thus flow out of the intra-discal space. The injection may also serve to coat areas adjacent to the disc, directly on the nerve roots and surrounding areas which serve to protect those areas from the effects of the leaking nucleus material. Sealing the fissures and bone end plates halts the leakage of harmful chemicals into the disc environment and prevents the initiation of foreign-body reactions towards the damaged disc by the immune system. Increasing the disc space relieves pressure on the nerve root. That is, as a result of the injection, an increase of the disc height occurs, which increases the spacing between lamina, and which in turn relieves pressure on the nerve roots on the lamina. For this application, supplementation of the fibrin sealant with growth factors may promote rehabilitation of the damaged tissues or the gradual replacement of the fibrin sealant with healthy tissue.

Use of the improved fibrin sealant composition may be better understood by reference to the following examples. These examples are representative and should not be construed to limit the scope of this invention or claims hereof. Unless otherwise indicated (example 3), corticosteroid is absent from the fibrin sealant being used in these examples and the procedures were conducted in the absence of a heating step of the nucleus fibrosus and annulus fibrosus and in the absence of a partial or total discectomy.

### EXAMPLE 1

### Fluoroscopic Transforminal Epidural Injection

With a patient in the prone position on the imaging table, a fluoroscope is positioned and adjusted to locate the intervertebral foramen of the affected nerve root. A curved 22ga. X 3.5″ needle is introduced after anesthetizing the skin and deep tissue. The needle is advanced under direct fluoroscopic vision to a position in the anterior epidural space. Positioning of the needle is verified by a lateral fluoroscopic view and by injecting contrast medium through the needle. Such positioning may or may not require further adjustment. If adjusted, location of the needle is once again verified. Advancement of the needle into the correct region may stimulate pain in a manner consistent with the initial complaint. Therefore, needle placement may also be verified by the patient's pain recognition. The epidural space is anesthetized with injectable anesthetic. The fibrin sealant of fibrinogen and thrombin (prior to clotting) is then introduced through the needle with continuous gentle pressure until the volumes of the dual syringe system are sufficiently depleted. The fibrin sealant then coats the nerve root and annulus and the needle is withdrawn. Patient observation and vital signs monitoring is performed for about 20-30 minutes following the procedure.

For this procedure, a sufficient volume of the fibrin sealant is injected to effectively hydro-dissect the area around the targeted nerve root. It is believed that due to the avascular nature of the epidural space, the absorption/degradation period is typically longer than that observed for open applications in regions with greater vascularity and exposure to room air at the time of application.

The ability of the fibrin sealant to seal annular fissures related to disc herniation offers a therapeutic benefit to patients. Chemical radiculitis, or inflammation of the nerve root, is known to be quite painful in some instances. It is believed that use of the fibrin sealant in the above described manner not only coats the nerve root, but also seals annular fissures surrounding the herniated disk. (See FIG. 1). As a result of the hydro-dissection of the area around the affected nerve root, the sealant also seals annular fissures from outside the annulus.

### EXAMPLE 2

### Fluoroscopic Guided Intra-Discal Injection

After sterile preparation, an introducer needle is advanced in oblique projection to a superior articular process. A curved spinal needle is advanced through the introducer needle into the disc. Both anterior-posterior and lateral fluoroscopic projections are used to confirm proper needle placement. If the needle placement needs to be adjusted, placement is again confirmed fluoroscopically. A contrast agent is injected to confirm needle placement. In patients with chemical radiculitis, the contrast agent can be observed to be leaking through the annular fissures and/or intra-discal pathology, thus permitting their identification. Once the needle is properly positioned in the intra-discal space, the fibrin sealant (or its components) is injected using a dual syringe system. The fibrin sealant is observed to force the contrast agent from the intra-discal space as it seals the annual fissures. Alternatively, the contrast agent is injected with the sealant. Alternatively, no contrast agent is used. The procedure seals the defects/fissures of the annulus fibrosus and stops the chemical leakage and facilitates regeneration within the disc.

### EXAMPLE 3

### Comparasion of the Injection of Fibrin Sealant to Injection of Fibrin Sealant Containing a Corticosteriod

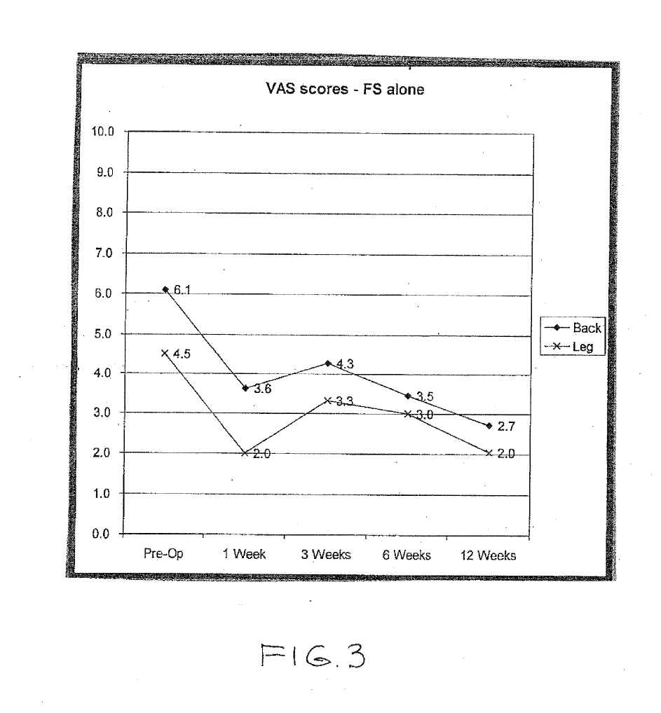

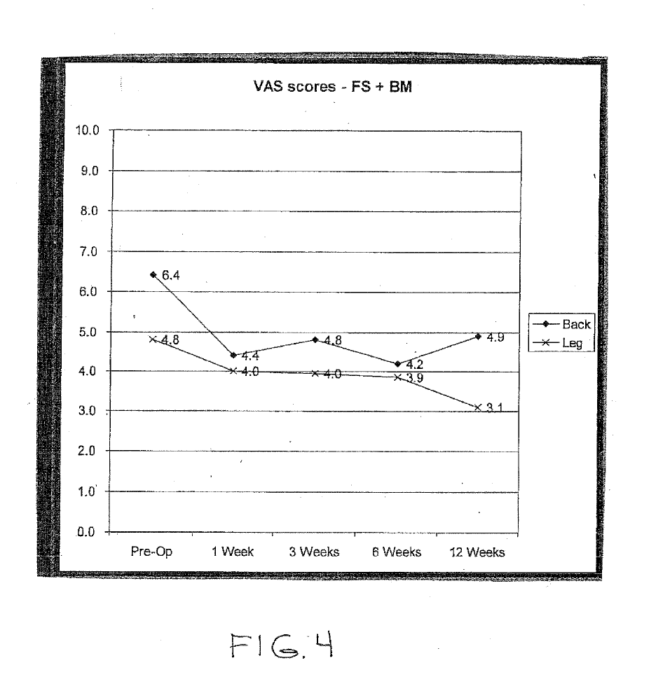

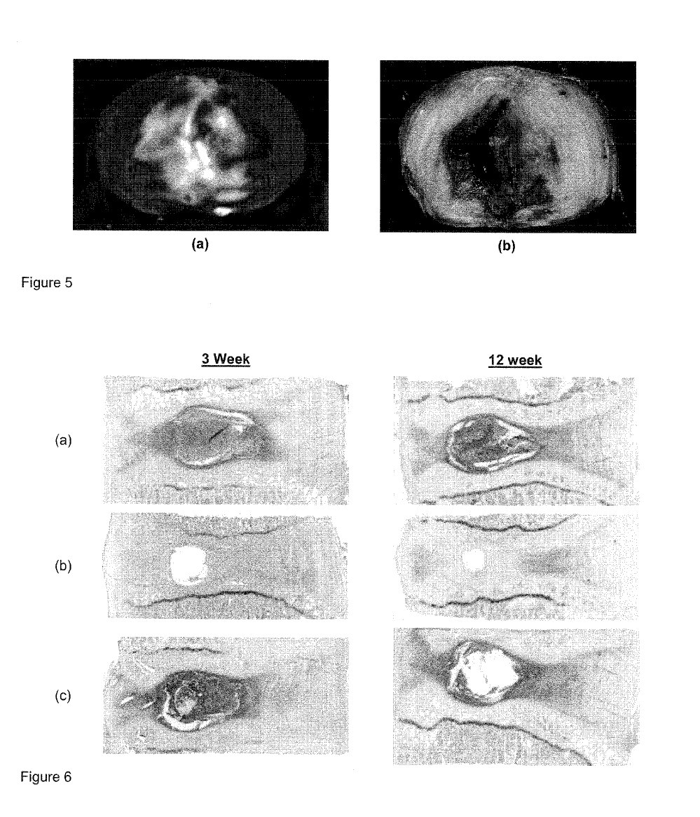

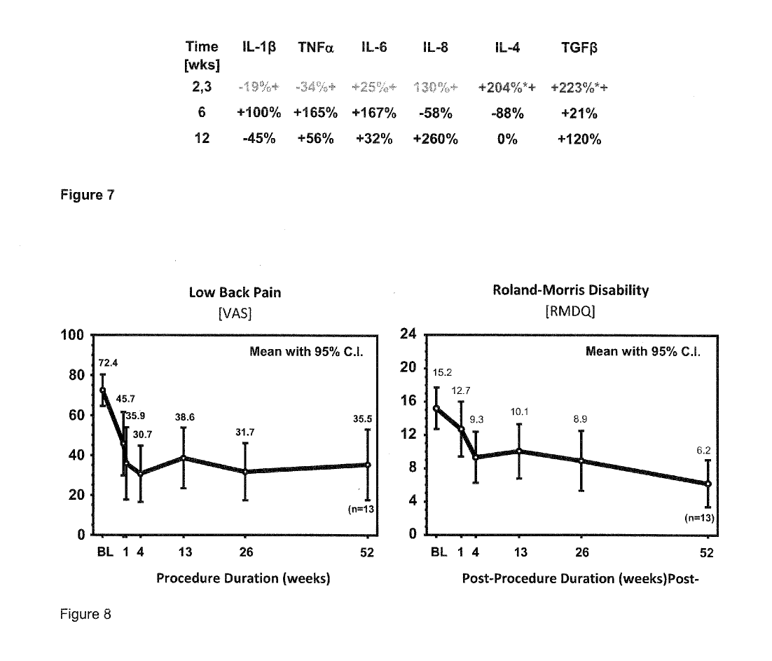

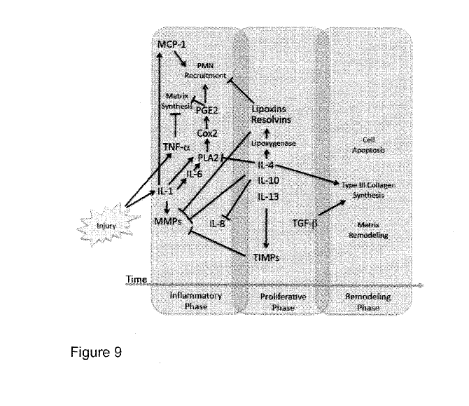

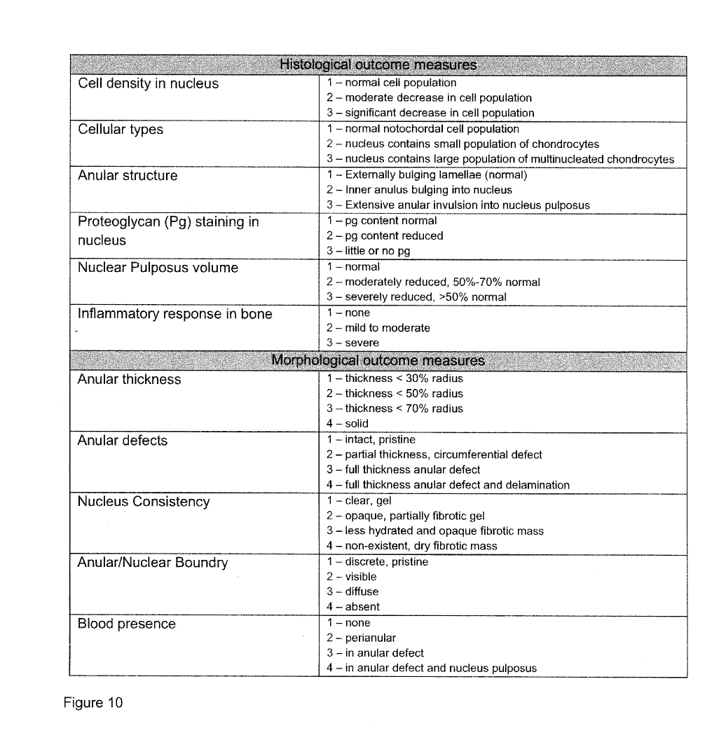

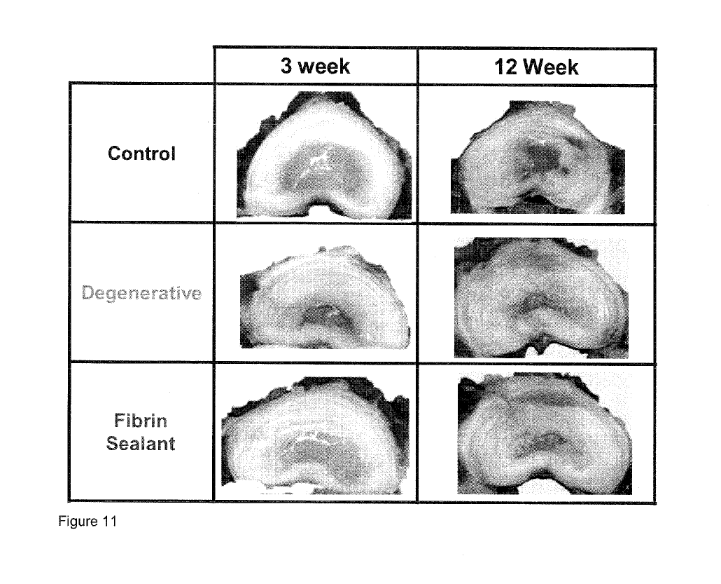

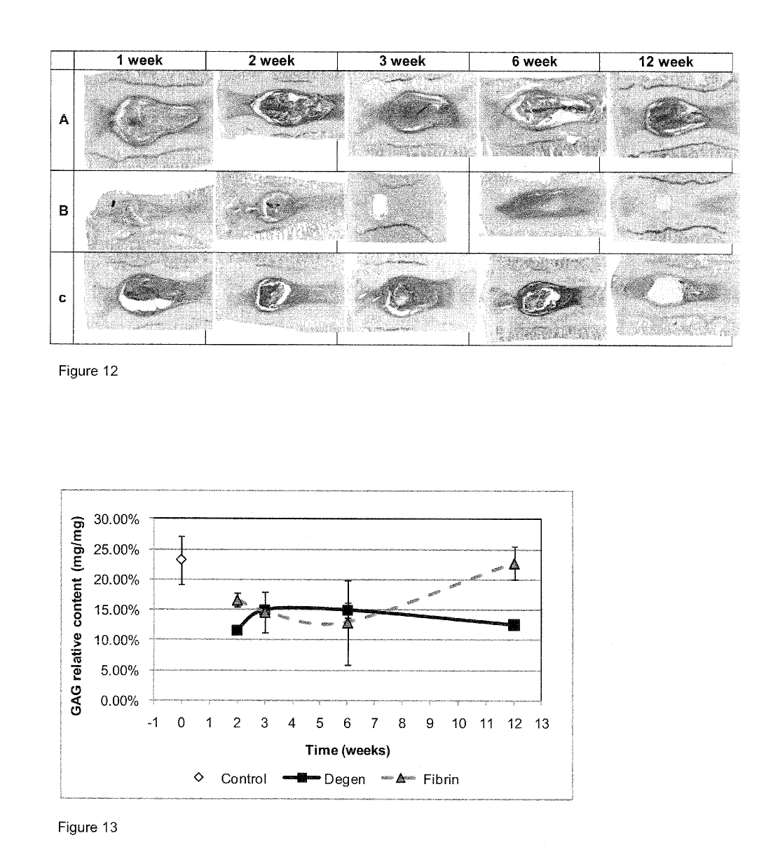

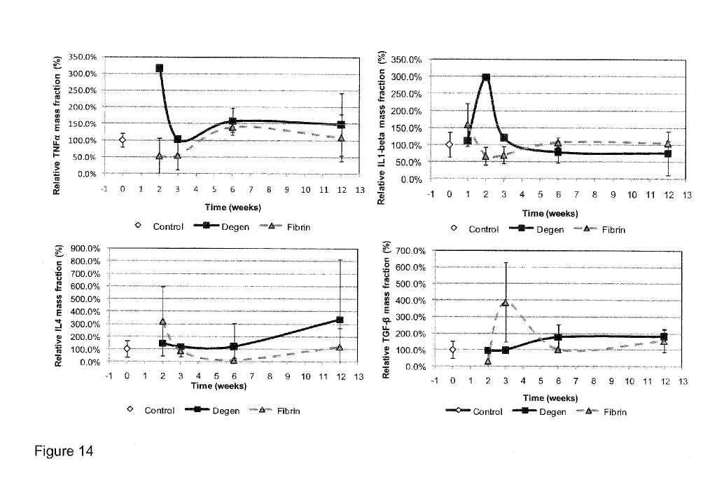

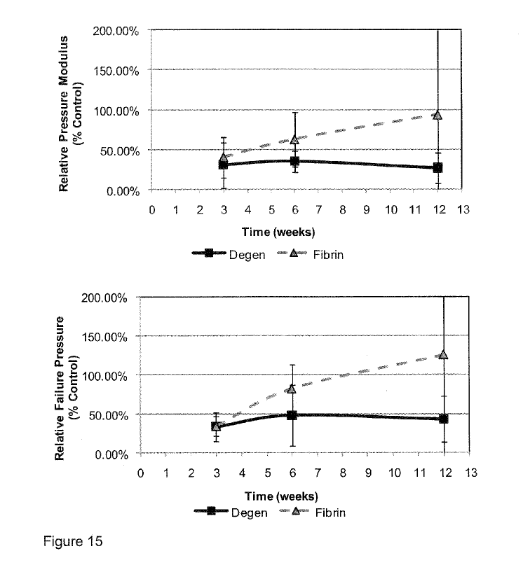

Twenty patients were split into two 10-patient groups. All of the patients suffered from pain due to disc degeneration caused by defects (fissures) in the annulus fibrosus. All of the patients had previously failed at least 6 months of traditional conservative therapy. Using the procedure in example 2, the first group of patients was intra-discally injected with fibrin sealant containing fibrinogen and thrombin. Using the procedure in example 2, the second group of patients was instra-discally injected with fibrin sealant containing fibrinogen, thrombin, and betamethasone (a corticosteroid). The corticosteroid was in the thrombin component. Each patient rated back and leg pain on a scale of 0 to 10, before and at predefined intervals following surgery. The results (VAS scores) are shown in FIGS. 3 and 4. As can be seen, the patients that were injected with fibrin sealant alone (without betamethasone) experienced superior pain relief to those patients that were injected with fibrin sealant containing betamethasone. It had been anticipated that the patients receiving injections that included betamethasone would experience superior results due to the action of the betamethasone to reduce inflammation. However, the opposite was observed. Indeed, the patients injected with fibrin sealant only experienced significantly improved pain relief, not only after one week from surgery, but especially 12 weeks after surgery. These results were surprising and unexpected.

In various embodiments of the invention, biologic solutions may enhance natural cellular tissue growth to replicate the unique composition, extracellular architecture and function of healthy tissues.

Because IVD degeneration may result from a catabolic imbalance in cellular function, various embodiments of the invention may include biologic solutions that enhance the anabolic repair and recovery of IVD tissues, which may provide therapeutic relief of back pain. Such solutions may include the use of anabolic growth factors, inhibitors of catabolic enzymes, cellular supplements and conductive tissue scaffolds.

Embodiments of the invention may be combined with various therapies such as, for example, providing pain relief by direct inhibition of nociception, blocking neural receptors, reducing the nuclear concentration of hyperalgesic inflammatory cytokines, or by eliminating nociceptive nerves. Such methods may include utilize intradiscal analgesics, intradiscal steroids, intradiscal neurotropic agents and intradiscal electrothermal and radiofrequency probes. Embodiments of the invention may be combined with more aggressive therapies such as, for example, lumbar fusion, and disc arthroplasty.

Embodiments of the invention may also include or be used in combination with various biologic solutions for therapeutic relief of CLBP. In contrast to conventional therapies designed for pain management, such embodiments may repair the damage associated with degeneration and discogenic CLBP and may inhibit catabolic processes associated with degeneration or promote extracellular matrix accumulation. These embodiments may include metabolic agents, viable cell supplementation and novel tissue scaffolds.

Regarding metabolic agents, embodiments of the invention may also include or be used in combination with intradiscal injection of metabolic agents that increase anabolic extracellular matrix accumulation. Such agents may include growth factors to stimulate IVD repair and can stimulate regenerative changes, including osteogenic proteins (OP-1, BMP-2, GDF-5, TGF-β, LMP-1), insulin-like growth factor-1 (IGF-1), platelet derived growth factor (PDGF) and basic fibroblast growth factor (bFGF). These growth factors can stimulate the proliferation and proteoglycan synthesis of anulus fibrosus and nucleus pulposus cells. In addition to anabolic stimulation, the metabolic agents may enhance the accumulation of extracellular matrix by down-regulating the synthesis of catabolic enzymes and reduce expression of hyperalgesic inflammatory cytokines, including IL-16, IL-6, and TNF-α. This may reduce hyperalgesia and provide direct relief of discogenic CLBP.

Use of such agents in or in combination with embodiments of the invention may overcome limitations associated with individual or isolated application of metabolic factors. Such limitations include that both viable cell populations and adequate vascular nutrition are required to support a measurable increase in metabolic activity. The clinical effectiveness of isolated application of metabolic agents may therefore be limited to patients with early disc degeneration because of the progressive reductions in vascular supply and cell population that occur with more advanced degeneration. In addition, multiple applications of metabolic agents alone may be required to prolong their benefit beyond a few weeks due to their short half life.

Regarding cells, embodiments of the invention may also include or be used in combination with cells therapies. Because degeneration significantly reduces the population of viable cells within the IVD, direct supplementation of the cell population may increase anabolic extracellular matrix accumulation.

Embodiments of the invention may include or be used in combination with implanted cells that produce large amounts of proteoglycans in the hypoxic, avascular environment of the degenerate IVD. Such cellular candidates include fibrochondrocytes and pluripotent mesenchymal stem cells isolated from autologous or allogeneic tissue sources.

Use of such cells in or in combination with embodiments of the invention may overcome limitations associated with individual or isolated application of cells such as, for example, the difficulty of isolating desirable cells from the nucleus pulposus because of the negative influence of degeneration on cell density and cell population. Other limitations include the functional performance of the isolated cells may be reduced with more extensive disc degeneration. Even if healthy disc cells are isolated, the development of cell therapies using autologous nucleus pulposus cells may be hindered by the significant difficulty involved in their expansion to clinically required cell populations. Allogeneic cells may be isolated in higher concentrations to satisfy these requirements, but their use may be limited by immunologic host tissue rejection.

Embodiments of the invention may include or be used in combination with autologous mesenchymal stem cells (MSCs), which may provide biologic repair of the degenerative IVD because they avoid the limitations of fibrochondrocytes. Pluripotent MSCs are most easily isolated in high concentrations from bone marrow and adipose tissue. These cells can differentiate within hypoxic environments into fibrochondrocytes that are phenotypically similar to those normally found in the IVD. MSCs can both survive and proliferate in the degenerative IVD and can also synthesize a proteoglycan-rich extracellular matrix similar to that found in healthy IVDs.

Use of such cells in or in combination with embodiments of the invention may overcome limitations associated with individual or isolated application of MSCs, such as the differentiation and anabolic functions of MSCs may be negatively influenced by the low pH and high concentration of inflammatory cytokines present within the nucleus pulposus of the degenerative human IVD. Other limitations include the uncertainty as to whether human endplate permeability is sufficient to support long-term increases in cell population and metabolism required for the regeneration of the IVD. Because of these limitations, supplementation of the IVD with only mesenchymal stem cells may offer clinical benefit only for patients with early stage degeneration.

Regarding tissue scaffolds, embodiments of the invention may also include or be used in combination with scaffold therapies that include biomaterials serving as conductive scaffolds to assist in the cellular repair and regeneration of damaged tissues. Such scaffolds may include biologic materials derived from natural components of the extracellular matrix, (i.e., collagen, proteoglycan and hyaluronic acid) and synthetic materials. Biologically derived materials may contain properties that facilitate cell attachment and function. With either type of material, the scaffold may temporarily provide an optimal microenvironment for cellular migration, proliferation and extracellular matrix formation. Also, the scaffold may degrade at the functional rate of tissue replacement to maintain structural continuity and physical function during cellular repair.

Use of such cells in or in combination with embodiments of the invention may overcome limitations associated with individual or isolated application of scaffolds such as, for example, the low population of cells and their limited nutrition within the healthy IVD, which may restrict the normal cellular synthesis of extracellular tissues. Such limitations include synthetic reductions associated with moderate disc degeneration, which make complicate whether the conductive benefits of tissue scaffolds will independently result in measurable improvements in extracellular matrix accumulation.

One embodiment of the invention includes an intradiscal therapy that combines a biologic tissue scaffold and metabolic agent. An example of such a biologic tissue scaffold and metabolic agent includes the intradiscal delivery of BIOSTAT BIOLOGX® Fibrin Sealant into the nucleus pulposus of a damaged IVD. BIOSTAT BIOLOGX Fibrin Sealant has a formulation, suitable examples of which are included herein, that combines the physical and conductive benefits of a three-dimensional fibrin scaffold with the anti-inflammatory benefits of aprotinin acetate.

An embodiment of the biologic tissue scaffold and metabolic agent (“biologic”) includes a human derived, biologic tissue sealant comprised of highly purified human fibrinogen and thrombin, two blood proteins involved in theformation of a fibrin clot. The components may produce highly porous, non-cytotoxic, fully resorbable, biologic tissue scaffolds. Fibrin tissue scaffolds have been used in normal vascularized tissue repair. They can protect and support the damaged tissue and conductively assist the progressive cellular repair processes associated with inflammation, cellular proliferation, tissue repair and tissue remodeling. The intradiscal delivery of fibrin sealant may compensate for the vascular limitations of the IVD that prevent normal clotting of fibrin within the internal disc disruptions associated with discogenic CLBP. Initially, its delivery to the IVD may provide temporary pain relief by protecting nociceptors in the anulus fibrosus to the inflammatory mediators concentrated within the nucleus pulposus. Aprotinin acetate is an antifibrinolytic agent which may be added to the fibrin sealant to slow the proteolytic degradation of the resulting fibrin matrix. Aprotinin acetate has antiinflammatory effects. As a result of its unique combination of constituent components, intradiscal application of embodiments of the biologic tissue scaffold and metabolic agent (e.g., BIOSTAT BIOLOGX) may reduce hyperalgesia, inhibit catabolic tissue degeneration, favor the accumulation of extracellular tissues and conductively facilitate healing of the internal disruptions of the anulus fibrosus as required for prolonged pain relief.

An embodiment of the biologic tissue scaffold and metabolic agent includes BIOSTAT BIOLOGX which may generally include Fibrinogen (67-106 mg/mL), Thrombin (400-625 IU/mL), Calcium Chloride (36-44 μmol/mL) and Aprotinin (2250-3750 KIU/mL). Other embodiments may generally include Fibrinogen (55-85 mg/mL), Thrombin (800-1200 IU/mL), and Calcium Chloride (35-45 mM). Additional embodiments may generally include Fibrinogen (90 mg/mL), Thrombin (500 IU/mL), Calcium Chloride (35-45 mM), and Aprotinin (1000 KIU/mL). Other embodiments may generally include Fibrinogen (80 mg/mL), Thrombin (250 IU/mL), Calcium Chloride (35-45 mM), and Aprotinin (1000 KIU/mL). Other embodiments may generally include different combinations within ranges such as Fibrinogen (50 to 150 mg/mL), Thrombin (200 to 1200 IU/mL), Calcium Chloride (40 μmol/mL) and Aprotinin (1000 to 3000 KIU/mL). Embodiments may not be completely pure and may contain smaller quantities of various other proteins which are not removed through a fractionation process (e.g. the fibrinogen component may contain total protein of 96-125 mg/mL of which 67-106 mg/mL is Fibrinogen). In particular, factor XIII may sometimes be added if the concentration is lower than 10-80 IU/ml. Factor XIII may play a role in clot strength and stability. Ca2+ ions may be included for the conversion of fibrinogen to fibrin and for the activation of factor XIII.

Embodiments of the biologic tissue scaffold and metabolic agent may be used in combination with delivery systems (e.g., as described herein, as described in U.S. Patent Application publication no. 2007/0191781, and/or Biostat® Delivery Device) to facilitate percutaneous delivery of the biologic to the IVD. The intradiscal delivery of commercial formulations of fibrin sealant through spinal needles is hindered by the rapid setting kinetics of fibrin sealants. The negative consequences associated with rapid setting kinetics include needle occlusion and inadequate intradiscal distribution of fibrin sealant. Embodiments of the delivery device eliminate these complications with a needle-in-needle construct that prevents mixing of the reactive components of the biologic until, for example, the terminal 2 cm of the intradiscal needle tip. Preclinical studies demonstrated that this delivery solution enhances the mechanical properties (strength, ductility and fracture toughness) and degradation resistance of the fibrin product. More importantly, the novel mixing and delivery system delays setting to enhance fibrin flow through the IVD and into the internal disc disruptions associated with discogenic pain. For enhanced safety, the delivery device may also contain an intradiscal pressure monitor to help avoid overpressurization of the damaged disc.

Preclinical cadaveric studies confirm the ability of the delivery device to uniformly distribute biologic tissue scaffold and metabolic agent within the IVD (FIG. 5). Comparisons with postdiscography CT images confirm a high correlation between the flow patterns of contrast and fibrin sealant within the IVDs. Thus fibrin is distributed in patterns corresponding to contrast through flow patterns of least resistance. Fibrin is observed to occupy free space within the nucleus pulposus and to penetrate and fill defects in the anulus fibrosis. Fibrin was detected within anular fissures to depths exceeding the outer third of their diameters where the nociceptive nerves associated with discogenic pain are concentrated.

Preclinical in vivo animal studies also provide strong evidence of the conductive and metabolic tissue healing benefits provided with embodiments of the biologic tissue scaffold and metabolic agent (e.g., BIOSTAT BIOLOGX Fibrin Sealant). The biologic was compatible with and significantly improved disc health in degenerative discs created by surgical damage (FIG. 6). Safety and cellular compatibility were demonstrated by both cellular proliferation and extracellular matrix synthesis in the nucleus pulposus and the lack of a provoked inflammatory response in the adjacent vertebral bodies. Conductive healing benefits were demonstrated by reduced fibrosis of the nucleus pulposus and promoted recovery of the proteoglycan content lost from nucleotomy damage.

Healing benefits were partially derived from the metabolic functions of embodiments of the biologic tissue scaffold and metabolic agent (e.g., BIOSTAT BIOLOGX Fibrin Sealant). In injured discs supplemented with BIOSTAT BIOLOGX, the cellular production of inflammatory cytokines associated with hyperalgesia and disc degeneration were reduced temporally as supported by the increased cellular production of the anti-inflammatory protein IL-4 (FIG. 7). Simultaneously, BIOSTAT BIOLOGX increased the cellular production of the essential growth factor protein, TGF-β, required for soft tissue healing. Additional preclinical studies demonstrated that BIOSTAT BIOLOGX Fibrin Sealant inhibited the cellular production of enzymes responsible for the degeneration of IVD tissues.

Data collected from a 15-patient FDA investigation support the safety and efficacy of BIOSTAT BIOLOGX Fibrin Sealant. The study demonstrated meaningful improvements for a majority of patients in both pain [Visual Analog Scale (VAS)] and function [Roland-Morris Disability Questionnaire (RMDQ)]. At the 6 month primary endpoint, the mean decrease in pain was 40.7 mm (55.6%) with 13/15 patients (88.6%) achieving at least 30% pain relief (FIG. 8). The mean improvement in RMDQ was 6.3 points (41.9%) with 11/15 patients (73.3%) achieving at least a 30% improvement in function. These benefits in pain and function were maintained through the 12-month extended follow-up visit.

Thus, in various embodiments of the invention intradiscal biologic therapies for chronic low back pain offer the hope of pain relief and functional improvements without the trauma of surgical intervention. Research has shown that metabolic agents and cellular approaches can slow disc degeneration in animal models.

Various examples embodiments of the biologic tissue scaffold and metabolic agent are addressed below. In a study surgically-denucleated porcine intervertebral discs (IVD) were injected with BIOSTAT BIOLOGX® Fibrin Sealant (FS), and the in vivo effects were characterized over time by histological, biochemical and mechanical criteria. The objectives were to test whether the intradiscal injection of FS stimulates disc healing by altering cytokine levels and increasing matrix synthesis.

This study was conducted in light of disc avascularity that prevents the deposition of a provisional fibrin scaffold that typically facilitates soft tissue repair. Poor disc wound healing leads to disc damage accumulation and chronic inflammation characterized by over production of pro-inflammatory cytokines and proteolytic enzymes.

Four lumbar IVDs from each of 31 Yucatan mini-pigs were randomized to: untreated controls; degenerative injury (nucleotomy); and nucleotomy plus FS injection. Animals were sacrificed at 1, 2, 3, 6 and 12 weeks post surgery. IVDs were harvested to quantify: 1) architecture using morphological and histological grading; 2) proteoglycan composition using DMMB assay; 3) cytokine content using ELISA; and 4) mechanical properties using quantitative pressure/volume testing.

Regarding results, there was progressive invasion of anular tissue into the nucleus of nucleotomy discs and concomitant reduction in proteoglycan content. By contrast, FS supplementation inhibited nuclear fibrosis and facilitated proteoglycan content recovery over time. FS discs synthesized significantly less TNF alpha than degenerate discs (66% vs. 226%, p<0.05) and had upregulation of IL-4 (310% vs. 166%) and TGF-β (400% vs. 117%) at 2-3 weeks post-treatment. At 3 weeks post surgery the denucleated discs were less stiff than controls (pressure modulus 779.9 psi versus 2754.8 psi, p<0.05), and failed at lower pressures (168.8 psi versus 519 psi; p<0.05). The stiffness and leakage pressure recovered to control values after 6 and 12 weeks, respectively in the FS-treated discs.

Regarding conclusions, the Fibrin sealant facilitated structural, compositional and mechanical repair of the surgically damaged IVD. These FS-derived benefits are due, at least in part, to its conductive scaffold properties, and metabolically active constituents such as thrombin, Factor XIII, and aprotinin acetate. Fibrin inhibited nucleotomy-induced progressive fibrosis of the nucleus pulposus. Fibrin sealant increased proteoglycan synthesis. Acute pro-inflammatory cytokine synthesis was decreased, and production of pro-resolution factors was increased including TGF-β and IL-4 in fibrin supplemented discs. These in vivo study results suggest that FS may provide healing and clinical benefits when injected into pathogenically degenerated IVDs. Thus, the in vivo porcine model was used to determine the influence of fibrin on: intervertebral disc architecture; proteoglycan composition; concentration of relevant biomarkers and mechanical properties. Fibrin injection blunted nucleotomy-induced increases in inflammatory cytokine production, promoted a more rapid recovery of mechanical properties, and enhanced proteoglycan matrix synthesis.

The early stages of intervertebral disc degeneration are defined by vascular supply reductions, cell death, proteoglycan depletion and nuclear dehydration. These degenerative changes provide evidence of the limited healing capacity of the IVD and justify the formation and progressive accumulation of radial and circumferential tears in the anulus fibrosus and concomitant mechanical property reductions that result in these highly stressed tissues. Because a subset of disrupted discs become painful due to neoinnervation as blood vessels and nerves penetrate with granulation tissue into these structural defects, novel technologies are desired to repair these defects, promote mechanical recovery and provide therapeutic pain relief.

Normal soft tissue repair is orchestrated by a set of cell mediated events distinguished by three functional phases: inflammation, proliferative repair, and tissue remodeling (FIG. 9). The inflammation phase is initiated by the release and formation of arachidonate-derived prostaglandins (e.g. prostaglandin E2; PGE2) leukotrienes, and chemokines (e.g. monocyte chemoattractant protein-1, MCP-1). These chemoattractants recruit cells and stimulate the synthesis of proinflammatory cytokines, such as IL-1, IL 6, IL-8, and TNF-alpha. These proinflammatory cytokines induce cell proliferation, chemotaxis and damaged tissue removal through the production of enzymes such as MMP-2, MMP-3, MMP-9, and MMP 13. The proliferative repair phase is characterized by fibroblast expansion, collagen secretion, angiogenesis, and granulation tissue formation mediated by profibrotic factors such as TGF-beta. The simultaneous secretion of anti-inflammatory cytokines (i.e., IL-4, IL-10 and IL-13) favors anabolic tissue accumulation by negating the effects of IL-1 and TNF-alpha reducing the synthesis of PGE2 and inhibiting the Cox2/PGE2 pathway. IL-4 and IL-10 also reduce PMN-mediated tissue degradation by stimulating the synthesis of anti-inflammatory lipid mediators (via lipoxygenase-mediated metabolism of arachidonic acid) such as lipoxins and resolvins. The tissue remodeling phase begins when the rates of matrix degradation and synthesis equalize. This may involve the production of Type III collagen that is gradually replaced by the stronger Type I collagen. There is also a reorganization of the extracellular matrix to optimize material properties and apoptotic removal of unnecessary cells.

In contrast, impaired healing is evidenced in degenerate and pathogenically painful discs by chronically elevated concentrations of PGE2, MCP-1, IL-1, IL-6, and IL-8 and TNF-α.13-16 The synthetic sources of these cytokines can be circulating inflammatory cells in the case of herniated discs or intervertebral cells in the case of contained disc degeneration. These cytokines upregulate the concentration and activity of several matrix metalloproteinases that are directly associated with proteoglycan depletion, nuclear dehydration, endplate sclerosis, and anular disorganization and disruption observed in the early stages of disc degeneration. Because several of these cytokines are hyperalgesic, discs with radial and circumferential tears may become painful if they interact with nociceptive nerves contained in the periphery of the anulus fibrosus. These cytokines may also stimulate discogenic pain when capillaries and nerves penetrate the anular disruptions during granulation tissue formation.

The purpose of this study was to determine if a metabolically active, biologic scaffold of fibrin sealant (BIOSTAT BIOLOGX® Fibrin Sealant) could enhance soft tissue repair following traumatic injury in an animal model. Numerous animal models have been investigated and developed for in vivo study of IVD injury and degeneration. Although they do not entirely duplicate the complex biological, structural, and mechanical situation of human intervertebral discs, surgically induced porcine models have proven highly useful for investigating the biochemical, structural and biomechanical changes that occur during disc degeneration. Several porcine studies have shown that acute disc injury stimulates an inflammatory cascade as evidenced by the secretion of elevated concentrations of PGE2, IL-1, IL-8 and TNF alpha and both structural and compositional degeneration. In contrast to humans, granulation tissue forms peripherally to the damaged anulus fibrosus. However, it is similarly rich in collagen, vascularized and highly innervated. It also contains similar concentrations of proinflammatory cytokines, growth factors, and matrix metalloproteinases that signal and regulate remodeling of the granulation tissue into fibrotic scar.

Fibrin sealants have been used in numerous surgical applications. The clinical benefits of fibrin sealant are partially derived from its ability to interact with matrix and cellular structures as an important component of normal wound healing. Their application relies on the reaction of two main components: the ‘sealant’ composed primarily of fibrinogen; and the ‘catalyst’ composed of thrombin and calcium chloride. When the sealant and catalyst are mixed, activated thrombin cleaves two peptides from fibrinogen leading to fibrin monomer formation. Hydrogen bonding of the monomers produces a nanoporous fibrotic scaffold within minutes. These structures have been demonstrated to promote the proliferation and migration of fibroblasts and stimulate collagen synthesis and granulation tissue formation. To optimize these conductive benefits, commercial fibrin sealant formulations with fibrinolysis inhibitors were required to preserve scaffold longevity in the aggressive proteolytic environment of the degenerate disc. BIOSTAT BOILOGX Fibrin Sealant (FS) was selected for use in this study because its fibrinolysis inhibitor, aprotinin acetate, may also benefit anabolic tissue accumulation by metabolically reducing inflammation. Given its formulation benefits, we hypothesized that FS may facilitate disc healing by reducing inflammation and stimulating cellular migration, proliferation and extracellular matrix formation.

The specific objectives of this study were to determine whether intradiscal delivery of FS can: a) reduce the soluble concentration or cellular expression of inflammatory cytokines; and b) stimulate the rate of extracellular matrix synthesis and the healing of the anulus fibrosus post surgical injury.

Thirty-one (31) Yucatan mini-pigs (4.6±1.6 years of age; Sinclair Research Center Inc.) were used in this study. Four lumbar intervertebral discs between levels L1/L2 and L5/L6 were exposed through a left side peritoneal approach. Among these, one disc served as an untreated control and the other three were denucleated using a 19G percutaneous discectomy probe (Stryker DeKompressor®). The probe facilitated the removal of approximately 0.75 ml of the nucleus pulposus within 1-3 minutes of operation. Some of the denucleated discs were used as untreated degenerative controls and the rest were supplemented with an average volume of 0.91 mL (±0.27 mL) BIOSTAT BIOLOGX Fibrin Sealant (FS) delivered using the Biostat® Delivery Device. FS was introduced slowly (˜0.2 mL/sec) until the delivery pressure stabilized at 100 psi.

At each study endpoint (1, 2, 3, 6, 12 weeks), animals were sacrificed and vertebra-disc-vertebra specimens were collected. The IVDs from each animal were assigned to either histological (n=3/group-time), biochemical (n=2/group-time) or biomechanical analysis (n=5/group time).

Discs for histology were isolated, fixed in formalin, decalcified (IED, Biocare), dehydrated and embedded in paraffin. Each sample was serially sectioned (6 micro meter thickness) in sagittal and coronal planes for respective assessments of general disc health and anular wound healing. All histological sections were stained with Safranin-O and Fast Green (FCF) (Sigma Chemicals).

Discs for biochemistry were sectioned along the mid-transverse plane and digitally photographed to document morphology. Soft tissues within 1 mm of the exterior anulus pulposus and the cartilaginous endplates were dissected, weighed and lyophilized. The residual solid mass was recorded as the organic tissue content. The difference in the dissected tissue mass and the organic tissue mass was recorded as the sample's hydration content. The residual organic tissue masses were enzymatically digested at 60 degrees C. for 16 18 hours with papain (Sigma Chemicals), centrifuged at 3000 rpm for 10 min and collected for biochemical analysis.

Immunoassays of the supernatants were performed to quantify the concentration of IL-1 beta, IL-4, IL-6, IL-8, TNF alpha (Pierce Searchlight, Woburn, Mass.) and TGF-beta (BioSource International, Camarillo, Calif.) using manufacturer recommended protocols. Proteoglycan content within the supernatants were analyzed using DMMB (1,9 dimethylmethylene blue) dye and duplicate plate reader (Molecular Devices, Sunnyvale, Calif.) assessments of spectrophotometric absorbance at 525 nm. Total cellular content was assessed by quantifying the content of deoxyribonucleic acid (DNA) using the PicoGreen fluorometric method [Molecular Probes].

Disc mechanical properties were assessed at 3, 6 and 12 weeks post surgery using quantitative discomanometry. The discs were tested by injection of a fluid [propylene blue dye (McCormic, 1%) and fluoroscopic contrast media (Hypaque)] via a syringe pump and primed PrecisionGlide® needle (19 Gauge) inserted into the center of the disc through a contra lateral approach (from surgical treatment). The fluids were delivered at a controlled rate (0.5 mL/min) until leakage was observed or the injection pressure limit was achieved (600 psi).

Anterior-posterior and lateral fluoroscopic x-ray images were taken of each spine before and immediately after injection of the contrast-dye mix. These images were used to measure disc volume and height. The average disc height was calculated by averaging the measurements obtained from the anterior, middle, and posterior portions of the IVD. Scar formation at the needle wound sites was recorded as: minor, moderate or significant.

The morphological and histological images were blindly scored by three reviewers using a semi-quantitative grading scheme (FIG. 10). Tissue composition values and cytokine levels were expressed in terms of mass fraction (total sample weight normalized by organic tissue mass), relative content fraction (total sample weight normalized by both organic tissue mass and average controls), or relative cellular synthesis (total sample weight normalized by both sample cellular DNA content and average controls). Plots of injection pressure (psi) versus normalized volumetric strain (ΔV/Vo—where Vo is the initial disc volume in mm3 measured from radiographs) were used to calculate the pressure modulus (psi), failure pressure (psi) and volumetric failure strain (%). These mechanical property values together with disc height (mm) were presented as averaged values and normalized ratios to intra-animal control values. Non-parametric statistical analysis (Exact Kruskal-Wallis analysis of ranks) was used to compare the measured differences between the control and treatment groups. When appropriate, post-hoc multiple pair-wise comparison tests (Dunn's) were performed to determine statistical differences at a significance of p<0.05. Multiple pair-wise comparisons with probabilities between 0.05<p<0.10 were defined as trends with near statistical significance 35. All statistical analyses were performed using SAS (Version 9.1).

Within untreated control discs, the border between the inner anulus and the nucleus pulposus was clearly distinct, surrounding a nuclear volume filled with a hydrated, gelatinous mass (FIG. 11). In the denucleated discs, there was a significant loss of distinction of this boundary (p<0.05) by 3 weeks post-surgery. The degenerative changes continuously and significantly worsened with time (p<0.05), as evidenced by gradual increases in anulus fibrosus thickness and reductions in the nucleus volume. In contrast, FS supplementation appeared to better preserve both the architecture of the anulus fibrosus and the consistency and appearance of material within the nucleus pulposus at all time points. In both the denucleated and FS supplemented discs, the needle wound sites were covered with perianular fibrotic scar that ranged in size from moderate to significant. However, the extent and size of scaring was reduced in the FS supplemented discs.

Untreated control discs had organized and uniform structures at all post-surgical durations (FIG. 12). The anulus fibrosus consisted of well-organized collagen lamellae and the nucleus pulposus contained a sparse population of notochordal cells dispersed in a proteoglycan enriched matrix. The thin cartilaginous endplates were continuous.

In comparison, the denucleated discs displayed moderate to severe degeneration as early as 2-3 week post-surgery. Structural degradation continued throughout the length of the study as evidenced by the significant (p<0.05) and gradual inward bulging of the inner and outer annuli and fibrotic tissue ingrowth into the nucleus. Nucleus degeneration was characterized by the gradual loss of its distinct proteoglycan enriched boundary layer, proteoglycan loss and reductions in volume. In addition, the notochordal cellular population was gradually replaced with multinucleated fibrochondrocytes by 3 weeks post surgical trauma. All these degenerative changes were statistically different from the untreated controls at 12 week post surgical damage (p<0.05).

In contrast, the FS supplemented discs had reduced signs of intervertebral degeneration that were not significantly different from the untreated controls at any post surgical duration (p>0.05; FIG. 3). FS supplementation better preserved proteoglycan content and nucleus volume (p<0.05 at 12-weeks) at all post surgical durations. It also appeared to better preserve lamellar orientation and reduce the extent of anular ingrowth into the nucleus pulposus. Residual amounts of FS were present histologically at all post-surgical durations without evidence of an inflammatory response.

In untreated control discs, proteoglycan content was 22.4 wt. % (±1.8 wt. %) of their organic tissue mass (FIG. 13). The proteoglycan content differences in the treatment discs were not statistically different than the untreated controls at any time point (p>0.05). However, all treatment discs presented with reduced proteoglycan content [13.3±2.4 wt. % (denucleated), 15.6±2.5 wt. % (FS)] at 2-3 weeks post-surgery as expected due to nucleotomy. Proteoglycan content remained consistently low in the denucleated discs at all time points. In contrast, FS supplemented discs gradually recovered in proteoglycan content achieving control content values (22.8±2.7 wt. %) by 12 weeks.

In comparison to intra-animal control values, there were no significant differences (p>0.05) in the mass fraction content of IL 1β, IL-4, IL-6, IL-8, TNF alpha and TGF-β in the treatments discs at any time point (p>0.5; FIG. 14). However, the denucleated discs contained elevated mass fractions of IL-1β (209%±124%), IL-4 (133%±19%), IL-6 (207%±23%), IL-8 (413%±300%) and TNF alpha (210%±150%) at 2-3 weeks post surgical damage. After 6 weeks, IL-4 and TNF alpha were the only cytokines that remained elevated (122%±187%, and 158%±41% respectively). At 12 weeks, the denucleated discs contained elevated mass fractions of IL-4, IL-6, IL-8 and TNF alpha. The denucleated discs also contained moderately elevated mass fractions of TGF-β after 6 weeks (177%±74%) and 12 weeks (182%±44%).

In contrast, discs supplemented with FS presented with reduced relative mass fractions of IL-1β (68%±2%) and TNF alpha (53%±1%) at 2-3 weeks post-surgical damage. The FS supplemented discs were simultaneously enriched in IL-4 (204%±166%) and IL-8 (155%±135%). After 12 weeks, IL1β, TNF alpha, IL-6 and IL-8 tended to remain elevated. FS supplemented discs contained a more highly elevated mass fraction of TGF-β mass fraction at 3 weeks (387%±240%). The elevated TGF-β content remained at 6 and 12 weeks, but to a lesser degree.

Relative to denucleated discs, FS supplementation significantly decreased TNFα (66% vs. 226%, p<0.05) and showed a trend in reduction for IL-1β (81% vs. 229%, p<0.1) at 2 and 3 weeks.

The mechanical properties of the untreated control discs did not vary with time (FIG. 15). In comparison, the denucleated samples presented with significant reductions in pressure modulus (779.9±577.3 psi, 31%; p<0.05) and leakage pressure (168.8±64.3 psi, 34%; p<0.05) at 3 weeks. Their average modulus (720.4±180.7 psi, 42%) remained significantly reduced (p<0.05) at 6 weeks. Their average modulus and leakage pressure were not statistically different (p>0.05) from untreated controls after 12 weeks, but the values remained consistently lowered (840.8±662.1 psi, 27% and 185±179.6 psi, 43%, respectively).

In comparison to the untreated control discs, FS supplemented discs presented with significant reductions in pressure modulus (1130.1±536.4 psi, 41%; p<0.05) and leakage pressure (185.5±44.6 psi, 34%; p<0.05) at 3 weeks post surgical damage. In contrast, FS supplemented discs gradually recovered normal values for pressure modulus (1360.2±536.4 psi, 93.7%) and leakage pressure (209.4±536.4 psi, 125%) by 12 weeks. The supplemented discs were not statistically different (p>0.05) from untreated control values in leakage pressure and pressure modulus by 6 weeks and 12 weeks, respectively.

The ability of FS to enhance disc healing after surgical nucleotomy was experimentally assessed by morphology, histology, matrix synthesis, cytokine secretion, and mechanical properties. When compared to untreated controls, intradiscal injections of FS resulted in preserved disc architecture, reduced secretion of pro-inflammatory cytokines, increased synthesis of pro-resolution factors (TGF-β, IL-4), and recovery the mechanical properties lost due to surgical nucleotomy and structural degradation.

Consistent with historical data, anular damage and nucleotomy triggered a proinflammatory response that was followed by progressive and irreversible structural degradation. The surgical damage was repaired with peripheral anular granulation tissue that evolved into perianular scar. Although the mechanisms of initial repair were similar, the amounts of perianular scar tissue surrounding the discs supplemented with FS were reduced. The observed reductions in scar formation are consistent with decreased cytokine production, suggesting that FS down regulated inflammatory processes associated with tissue repair. This observation is supported by published evidence that hypertrophic scar formation is clinically associated with chronically elevated concentrations of pro inflammatory cytokines.

Structural degradation of the denucleated porcine discs was evidenced by the significant inward bulging of the anulus fibrosus that became progressively worse over time. The structural degradation of the nucleus was manifested by a reduction in proteoglycan content. These observations are consistent with prior studies that report damaged and degenerated IVDs exhibit decreases in the content and synthesis of proteoglycans and the accumulation of aggrecan degradation fragments. The data indicates that there is a moderate increase in proteoglycan content at three weeks (15.0 wt. %, p>0.05). This short-term reaction likely reflects a natural repair mechanism as the acute response to anular damage in animal models involves increased synthesis of proteoglycan. Also, gradual replacement of notochordal cells by multinucleated fibrochondrocytes was observed. These cells are likely derived from inner anulus tissue that progressively replaces the nucleus pulposus. In contrast, FS supplementation reduced inward anulus bulging and disorganization. This is likely due to the mass effect of FS that prevents acute structural collapse and tissue stress redistribution. FS also preserved the volume of the nucleus pulposus at all post surgical durations and enhanced proteoglycan content recovery.

Pro-inflammatory cytokines such as TNF alpha, IL-1β, IL-6, IL-8 and prostaglandin E2 are known to be elevated during disc degeneration. These cytokines are usually localized to the anulus and the nucleus near the damaged regions, and trigger matrix remodeling by stimulating the secretion of MMP-3 and MMP-13.44 Consistent with this, we observed a transient increase in the inflammatory cytokine content two weeks post surgical trauma in the denucleated discs. This temporal increase in TNF alpha, IL-1β and IL-6 are characteristic of a normal traumatic wound response and is consistent with prior reports of the wound response for articular cartilage. These pro-inflammatory molecules up-regulate the secretion of matrix metalloproteinases that promote catabolic tissue degradation and are likely responsible for the architectural features of degeneration noted histologically at longer time points.

Intradiscal injections of FS significantly reduced the production of the proinflammatory cytokine TNF alpha (p<0.05). In addition, FS supplementation appeared to stimulate the secretion of the pro-resolution cytokine IL 4 at 2-3 weeks. These effects may be due to two mechanisms. First, fibrin sealant is known to chemically bind cytokines due to its high structural surface area when Factor XIII is present. Chemical binding can reduce the local concentration and availability of active cytokines and thereby inhibit their biological function. However, these effects are due, at least in part, to the metabolic activity of its anti-fibrinolytic additive, aprotinin acetate. Aprotinin acetate is a potent anti inflammatory agent that upregulates the synthesis of anti-inflammatory cytokines, including IL-4 and IL-10, which inhibit the synthesis of TNF alpha, IL-1β, IL-6, and IL 8. Additionally, IL-4 down regulates the synthesis of MMP-2 cytokines reducing catabolic collagen degradation. The moderately up-regulated content and synthesis of IL-8 that occurred following surgical damage FS supplementation may be due to another FS component, thrombin, which is a most potent activator of IL-8.

TGF-β stimulates an anabolic response characterized by the enhanced cellular synthesis of both collagen and proteoglycan. We observed that FS supplementation tended to increase TGF-β at three weeks post-surgery, which may be responsible for enhanced proteoglycan synthesis that occurred between six and twelve weeks post denucleation.

Surgical denucleation caused a reduced pressure moduli and lower bursting pressure (p<0.05). These inferior mechanical properties correlated with architectural and biochemical changes that are consistent with prior reports: degeneration leads to both disorganization and tearing of the collagen fibers in the anulus and proteoglycan content depletions at the border of the anulus and within the nucleus. Even though samples supplemented with FS possessed significantly reduced (p<0.05) pressure moduli and failure pressures at 3 weeks post surgical damage, by 6 and 12 weeks both values returned back to control levels. The combined benefits of intervertebral FS supplementation maintained nuclear volume and enhanced the recovery of proteoglycan content as required to restore normal load-bearing dynamics and reduce the negative mechanical consequences of disc degeneration.

Several animal models (including sheep, pig, rabbit, dog, etc) have been used in attempt to understand disc degeneration in humans, each with advantages and disadvantages. One of the limitations in this current study is the presence of nothocordal cells in porcine IVDs, as opposed to nuclear chondrocytes in humans. These cellular differences may affect FS persistence and cell signaling. Also, human discs are larger and represent a more significant ischemic environment that may inhibit the ability of intervertebral cells to synthesize extracellular matrix in response to FS exposure.

Despite these limitations, the data highlight the wound healing response triggered by nucleotomy, and suggest that FS may assist in the anabolic recovery of the extracellular matrix content leading to architectural maintenance. The observed effect of FS in the porcine model suggests potential clinical benefits for degenerate human IVDs.

It is envisioned that the present invention may be used to address various conditions through use of the fibrin sealant in a manner similar to that described in the examples above. Discussion of this invention referenced particular means, materials and embodiments elaborating limited application of the claimed invention. The invention is not limited to these particulars and applies to all equivalents. Although this invention has been described above with reference to particular means, materials and embodiments, it is to be understood that the invention is not limited to these disclosed particulars, but extends instead to all equivalents within the scope of the following claims.

What is claimed is:

1. A composition for forming a resorbable tissue scaffold for enhancing tissue repair of at least one fissure in the anulus fibrosus of a painful intervertebral disc comprising: fibrinogen, thrombin, and calcium chloride.

2. The composition of claim 1 that includes a fibrinolysis inhibitor.

3. The composition of claim 2 wherein the fibrinolysis inhibitor is aprotinin.

4. The composition of claim 2 wherein the fibrinolysis inhibitor is aprotinin acetate.

5. The composition of claim 2 wherein the scaffold is one of conductive and inductive.

6. A method of enhancing tissue repair of at least one defect in the anulus fibrosus of an intervertebral disc comprising:
injecting fibrinogen, thrombin, calcium chloride, and aprotinin into a disc;
wherein (a) the fibrinogen and thrombin react to form a fibrin clot within at least a portion of at least one defect in the anulus fibrosus, (b) the fibrin clot includes a resorbable scaffold for tissue formation, and (c) the aprotinin downregulates the cellular synthesis of inflammatory cytokines and upregulates the syntheses of tissue growth factors to promote tissue repair.

7. The method of claim 6, wherein the aprotinin includes includes aprotinin acetate.

8. The method of claim 6, including injecting the fibrinogen, thrombin, calcium chloride, protein, and aprotinin into a soft tissue defect included in the disc.

9. The method of claim 6, including reduce cellular synthesis of inflammatory cytokines and proteolytic enzymes based on the aprotinin.

10. The method of claim 6, including injecting a metabolic agent that promote anabolic tissue formation.

11. The method of claim 6, including injecting a metabolic agent that stimulates cellular synthesis of anabolic growth factors.

12. A method of enhancing tissue repair of at least one defect in the anulus fibrosus of an intervertebral disc comprising:
injecting fibrinogen, thrombin, calcium chloride, and a fibrinolysis inhibitor into a disc;
wherein (a) the fibrinogen and thrombin react to form a fibrin clot within at least a portion of at least one defect in the anulus fibrosus, (b) the fibrin clot includes a resorbable scaffold for tissue formation, (c) the fibrinolysis inhibitor downregulates the cellular synthesis of inflammatory cytokines and upregulates the syntheses of tissue growth factors to promote tissue repair.

13. The method of claim 12, wherein the fibrinolysis inhibitor includes one of aprotinin and aprotinin acetate.

14. The method of claim 13 including forming a tissue matrix for enhancing repair within the at least one fissure in the anulus fibrosus.

15. The method of claim 13 wherein the disc is degenerated and prone to rapid degredation from elevated concentrations of proteloytic enzymes and low pH present within the disc.

16. The method of claim 12, wherein the fibrinolysis inhibitor includes aprotinin, the fibrinogen is between 60 and 110 mg/mL, the thrombin is between 390 and 640 IU/mL, the Calcium Chloride is between 30 and 50 μmol/mL, and the Aprotinin is between 2200 and 3900 KIU/mL.

17. The method of claim 12, wherein the fibrinolysis inhibitor includes aprotinin configured to upregulate the syntheses of tissue growth factors for longer than 4 weeks after injection into the disc.

18. The method of claim 12, wherein the fibrinolysis inhibitor includes aprotinin configured to downregulate cellular synthesis of inflammatory cytokines for longer than 2 weeks after injection into the disc.
